(19) United States
(12) Patent Application Publication
(10) Pub. No.: US 2008/0200994 A1
(43) Pub. Date: Aug. 21, 2008

(54) Detector and Stimulator for Feedback in a Prosthesis

(76) Inventors: J. Edward Colgate, Evanston, IL (US); Julio Santos-Munne, Glenview, IL (US); Keehoon Kim, Evanston, IL (US); Alex Makhlin, Chicago, IL (US); Michael Peshkin, Evanston, IL (US); Doug Schwandt, Redwood City, CA (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(21) Appl. No.: 11/677,564

(22) Filed: Feb. 21, 2007

(51) Int. Cl. A61F 2/00

(52) U.S. Cl. 623/24

(57) ABSTRACT

An apparatus and method for conveying sensory information from a distal location on a prosthetic limb, to a proximal location on the body of the wearer. The apparatus comprises a detector for mounting in a prosthesis and a stimulator for engaging the skin of the prosthesis wearer. Tactile, haptic and other information including surface-normal force, shear force, vibration, and/or temperature are sensed, conveyed, processed, and displayed, such that the wearer of the prosthetic has improved sensation and awareness from distal parts of a prosthetic, such as a fingertip.

### BACKGROUND

The invention pertains generally to afferent feedback (also known as sensory feedback) for prosthetic limbs.

Myoelectric prosthetic limbs use EMG signals generated by muscles of the wearer to control actuation of the prosthesis. Typically, the wearer of the prosthetic limb must learn to control operation of the prosthesis by training another, remaining set of muscles. The effort required to do this is demanding, difficult and time consuming. However, a recently discovered technique called target muscle reinnervation may avoid the difficult retraining and using of prosthetic limbs. Nerves leading to the missing limb, which were once used to transmit signals controlling its movement and receiving sensory information, sometimes remain after amputation. These nerves can be used to control a myoelectric prosthetic limb by anastomizing them to muscles no longer being used. When the wearer thinks about moving the missing limb, the reinnvervated nerves actually cause remaining muscles to twitch, which generates the EMG signal.

The process of reinnervating nerves from the missing limb also leads to reinnervation of cutaneous afferents and, possibly, kinesthetic afferents (muscle spindles and golgi tendon organs). Because the same nerve reinnervates both muscles and afferents, collocation is achieved naturally. Targeted reinnervation thus also provides the advantage of collocated muscles and afferents.

There have been attempts to convey sensory information from distal parts of a prosthetic to proximal parts of the human body, but none in connection with targeted reinnervation. Examples of feedback include mechanical conveyance of force, such as shown in U.S. Pat. No. 3,751,733, hydraulic conveyance of force such as shown in U.S. Pat. No. 4,808,187, and electromechanical conveyance of force, such as shown in U.S. Pat. No. 5,888,213. Nevertheless, most prosthetic limbs lack significant afferent feedback. For example, prosthetic hands lack the sense of touch. Wearers of prosthetic hands therefore have no direct way to sense tactile quantities such as grip pressure, surface roughness, surface warmth/coolness, and so on.

### SUMMARY

The invention concerns generally the feedback of tactile or haptic sensations of various forms from a distal location—for example, on a prosthetic—to locations on the wearer, overcoming one or more of the disadvantages of the prior art. Tactile or haptic sensations include, for example, one or more of temperature, vibration, and shearing and normal forces sensed by the prosthetic.

The transmitted sensory information is preferably presented in the same mode as it was sensed. For example, pressure forces that are measured distally are presented as pressure forces applied to the skin surface of the wearer and sensed vibration is presented as vibration. The sensory information as presented to the wearer may be applied at any sensate surface of the wearer's body. It may be presented inside the socket which attaches the prosthetic to the body. The term socket refers to the attachment system of the prosthetic, regardless of its shape. For instance a vest-like socket might be used to attach a whole-arm prosthetic. The sensory information might also be presented to the wearer at some other location, not in the socket.

One advantage of the invention is that it is well-suited for use with targeted reinnervation. Placing a stimulator (also referred to as a tactor) next to skin reinnervated with nerves from a missing limb, allows the wearer the possibility of experiencing the afferent sensations from a prosthetic as if they originate in the corresponding part of the missing limb itself.

The teachings of various aspects of the invention, in their preferred form, are explained in context of exemplary implementations of detectors (also referred to as receptors) for detecting tactile or haptic sensations and stimulators for transmitting the sensations to the skin of a person. The invention is not, however, limited to the details of these examples. The boundaries of the invention are limited solely by the appended claims.

As described in more detail below, one exemplary implementation of a receptor or detector preferably includes a three-axis force sensor. The force sensor is, in the example, preferably implemented by strain gauges affixed to a flexure. However, other force sensing mechanisms could be substituted. Inclusion of an accelerometer, for example, a multi-axis MEMS accelerometer, offers additional advantages. It is further preferred that the accelerometer is closely coupled to a light (low mass) but rigid or hard structure which serves the purpose of a fingernail or mechanically of a stylus, and aids in the exploration and detection of vibrational signals for a surface being touched or explored with the detector. The receptor or detector also preferably possesses an anthropomorphic shape. For example, if it is to be used in prosthetic hand, it preferably has the shape of a fingertip.

A stimulator, as shown in the exemplary implementations, preferably possesses a flat aspect and an activated tip moving largely perpendicular to the flat aspect when contacting the skin of the wearer of the prosthetic. The flat aspect is advantageous, and thus preferred, because it allows mounting the stimulator low and close to the socket, so that it is more comfortable to the wearer and protrudes little. It may be recessed into the socket. The stimulator preferably moves along at least two axes, one perpendicular to the skin surface, which may be denoted pressure, and one parallel to the skin surface, which may be denoted shear. Another axis of shear might could be added. One advantage of this example is that it can convey vibrations sensed by the detector to the skin. In the illustrated embodiments, two axes of motion are driven by two electric motors through gear trains and a linkage mechanism. Although the illustrated embodiment offers certain advantages, other types of actuators could be substituted.

The stimulator tip may also further adapted to include a heating and/or cooling unit such as a Peltier device. It is also possible to add an actuator, for instance as a voice-coil actuator, to the tip, in order to deliver vibrations of a higher frequency than the motors and transmission can accommodate.

In the exemplary embodiments information from the receptor or detector is transmitted as electrical signals. Sensors in the receptor can include, for example, strain gauges, accelerometers, miniature microphones, and thermometers. There are many varieties of these sensors and many are suited to small scale, low power, robustness, and other conditions of use of a prosthetic. While other modes of transmission of the information from these sensors are possible, including mechanical cables or linkages, hydraulic or pneumatic tubes, RF/wireless, fiber-optic, etc., electrical signals conducted by wiring are presently preferred. The signals may be analog levels, or may be multiplexed or conveyed as a data stream. Electrical conveyance of the signals from the receptor to the stimulator affords the opportunity of signal processing. The signal processing may be used to create mapping between receptor signals and the stimulator actions to make best use of the range of sensations available at the stimulation site. Each type of sensation such as pressure, shear, or temperature may have an independent mapping. In a preferred embodiment the signals are compressed in dynamic range, limited in amplitude, dead-banded, bandpass filtered, and filtered in frequency according to an equalization curve.

In applications involving a socket worn on the chest, for example, the chest skin and muscle of the wearer moves to some extent relative to the socket. One example of a stimulator described below is mounted to a socket so that it can stay in contact with the wearer's chest despite relative motion of the socket and the chest. A flexible coupling allows mounting of larger parts of the stimulator, for example its motors, to the socket, while other parts remain in contact with the wearer's chest at appropriate levels of force.

Although the invention is used to advantage in providing both detection of sensory information and presentation of it to the wearer of a prosthetic, these functions may be separated and used singly, for instance in some cases a synthetic (e.g. computer generated) sensation might be presented to the wearer via the stimulator, without or in addition to sensations originating at the detector. Similarly, the detector might be used alone without the presentation of its outputs by a stimulator. The invention and/or various aspects of it could be implemented in other applications in which it is desired to detect, convey, and display tactile or haptic sensations.

### BRIEF DESCRIPTION OF THE DRAWINGS

FIG. 1a is a sketch illustrating basic system components of an afferent feedback system for a prosthetic limb.

FIG. 1b is an isometric view illustrating two possible methods of attaching a haptic stimulator to a socket.

FIG. 1c is a schematic diagram of a detector and stimulator.

FIG. 2a is an exploded view of an embodiment of a detector.

FIG. 2b is an exploded view of a second embodiment of a detector.

FIG. 2c is an isometric view of the detector of FIG. 2b.

FIG. 3a is an isometric view of an embodiment of a two (2) degree of freedom stimulator.

FIG. 3b is an isometric view of a linkage of the stimulator of FIG. 3a.

FIG. 4 is an isometric view of an embodiment of a one (1) degree of freedom stimulator.

FIG. 5a is an isometric view of a second embodiment of a one (1) degree of freedom stimulator that can be mounted remotely.

FIG. 5b is a second embodiment of a linkage mechanism of the tactor embodiment illustrated in FIG. 5a.

FIG. 5c is an exploded view of the stimulator tip of FIG. 5b.

FIG. 6a is an isometric view of a second embodiment of a two (2) degree of freedom stimulator.

FIG. 6b is an exploded view of a the tactor illustrated in FIG. 6a.

FIG. 7 is a block diagram of a control signal processing.

### DETAILED DESCRIPTION OF EXEMPLARY EMBODIMENTS

In the following description, like numbers refer to like elements.

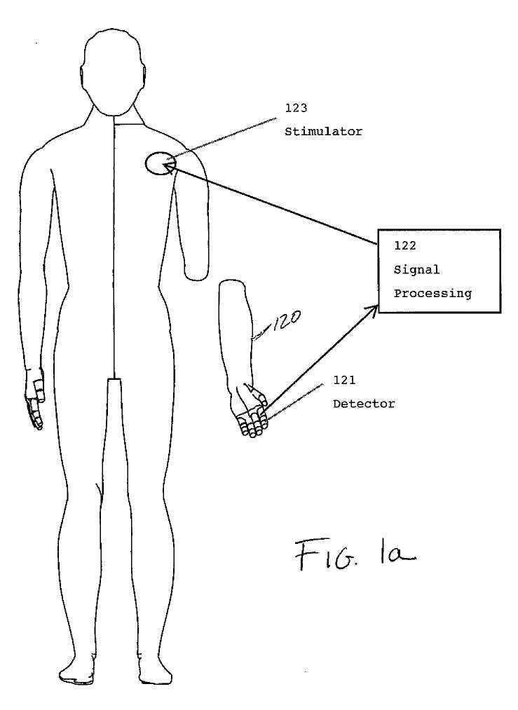

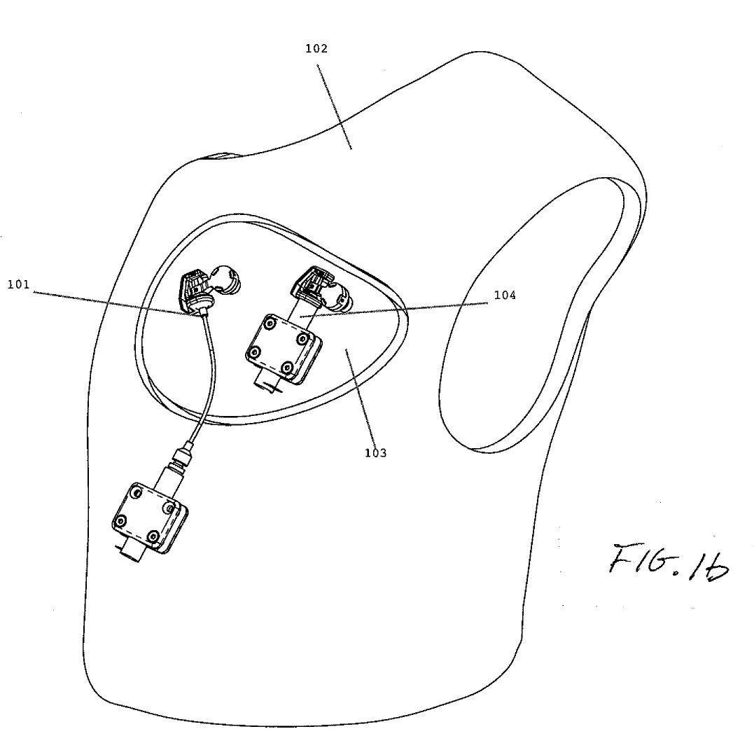

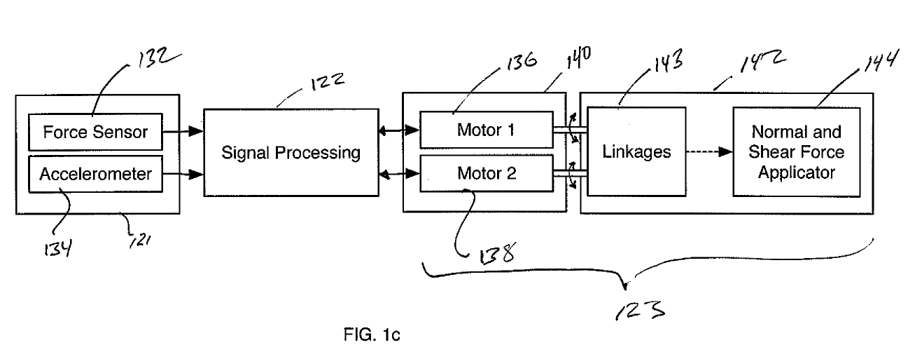

Referring to FIG. 1a, detector 121 receives signals from the environment that is touched by a prosthetic part, perhaps a fingertip. In this example, the detector is mounted in the finger tip of a prosthetic arm 120. The detector includes a plurality of sensors, for example sensors for sensing forces or pressures in several axes, for instance pressure normal to the surface and shear forces tangential to it, temperature or thermal conductivity, and/or vibration, measured for example by a single or multiple axis accelerometer. These signals are conveyed and processed by a signal processing circuitry 122 (which can be spatially distributed if desired), which modifies and combines the signals. The signals are transmitted to a sensate part of the wearer's body, in this case a region of the chest, and displayed by a stimulator 123. The stimulator can apply to the skin one or more of the senses of temperature, vibration, pressure, and forces in various directions. Thus the sensations that the wearer feels are derived from corresponding sensations originating in part of the prosthetic limb, here the fingertip. If the part of the body that receives the sensations has been surgically reinnervated by the technique called targeted reinnervation, the wearer may experience the sensation as if they originated in the lost part of the body, here the fingertip.

Referring to FIG. 1b, to alternate embodiments a stimulator are illustrated. Stimulator 104 is mounted directly, and stimulator 101 is mounted remotely, to pad 103. Pad 103 is typically preloaded against the wearer's skin by way of straps originating on socket 102. In this case, socket 102 is a type used for a prosthetic arm (not shown). Use of both stimulators is not required. Stimulator 101 is an alternate embodiment that allows for remote mounting of the stimulator's larger components on socket 102 and mounting stimulator tip on pad 103.

Referring now to FIG. 1c, detector 121 preferably includes at least one force sensor 132 and at least one accelerometer 134. The force sensor senses forces along at least two, and preferably three, axes with respect to the detector, with one of the axes normal to the surface of the prosthetic in which the detector is mounted. As described in more detail below, one dimensional or multi-dimensional force detectors can be used. Sensing forces along at least two axes allows detection of forces that are normal to the detector and shearing forces in at least one dimension. Sensing forces along at least one other axis allows detection of shearing forces in two dimensions. The accelerometer senses acceleration along at least one of the axes, and preferably two axes One-dimensional or multi-dimensional accelerometers can be used. The force detectors are used to sense static forces as well as low frequency vibrations. The accelerometers are used to sense higher frequency vibrations.

Signals from the force detectors and accelerometers are processed by signal processing circuitry 122 to generate output signals for driving preferably at least two motors 136 and 138. The signal processing circuitry acts as a controller for stimulator 123, and preferably receives feedback information from stimulator 123. The circuitry can be implemented in any way desired, and need not be dedicated to just processing signals from the detector. For example, the signal processing circuitry can be implemented using analog circuits, a digital signal processor, or combinations of the two. Furthermore, it can be incorporated in the detector or stimulator, distributed between them or other components.

The motors are indicated as being of stimulator 123, but may be mounted in a structure 140 separate from the stimulator head 142, as illustrated in FIG. 1b. The linkages 143 couple the rotational motion of the motors to motion of a skin engaging element 144 that applies to the skin a normal force and a shearing force in at least one dimension in response, at least in part, to the forces sensed by the detector. When used in combination with target muscle reinnervation, the detector, stimulator, and signal processing preferably attempt to recreate in the reinnervated area sensations in the user that mimic what the user would have felt with the user's missing limb. The output of the stimulator does not necessarily mimic exactly the forces and vibrations sensed by the detector.

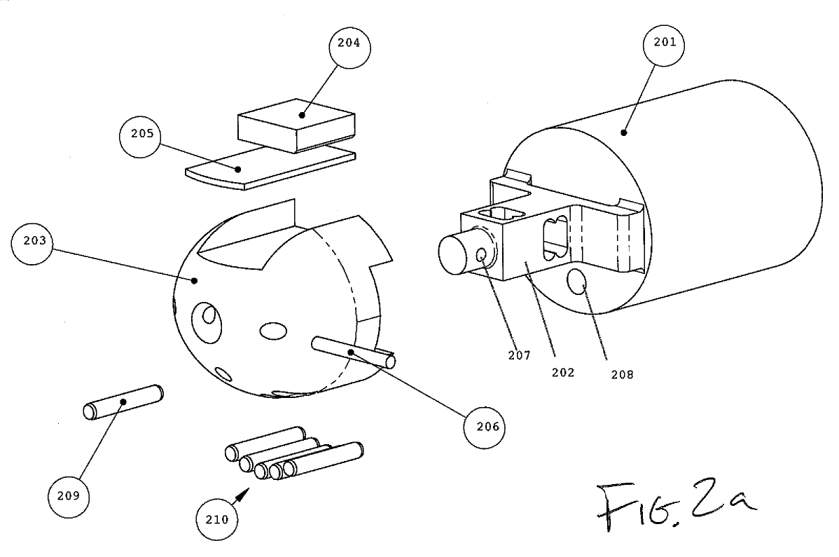

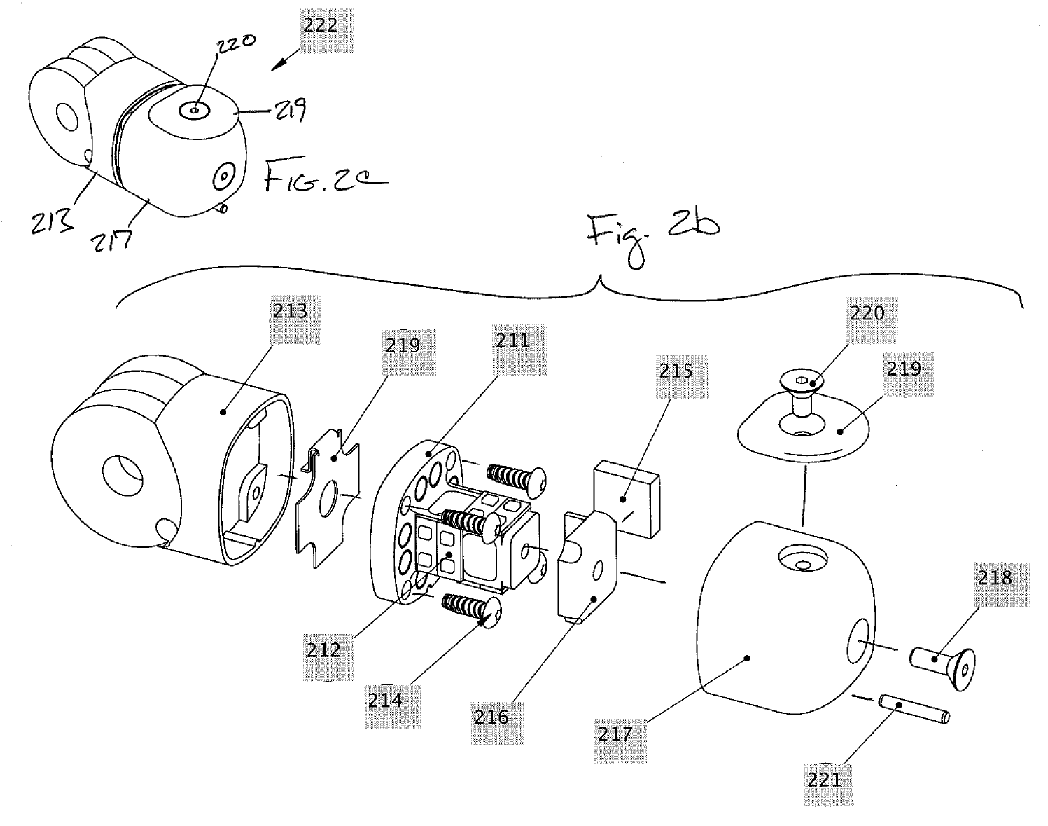

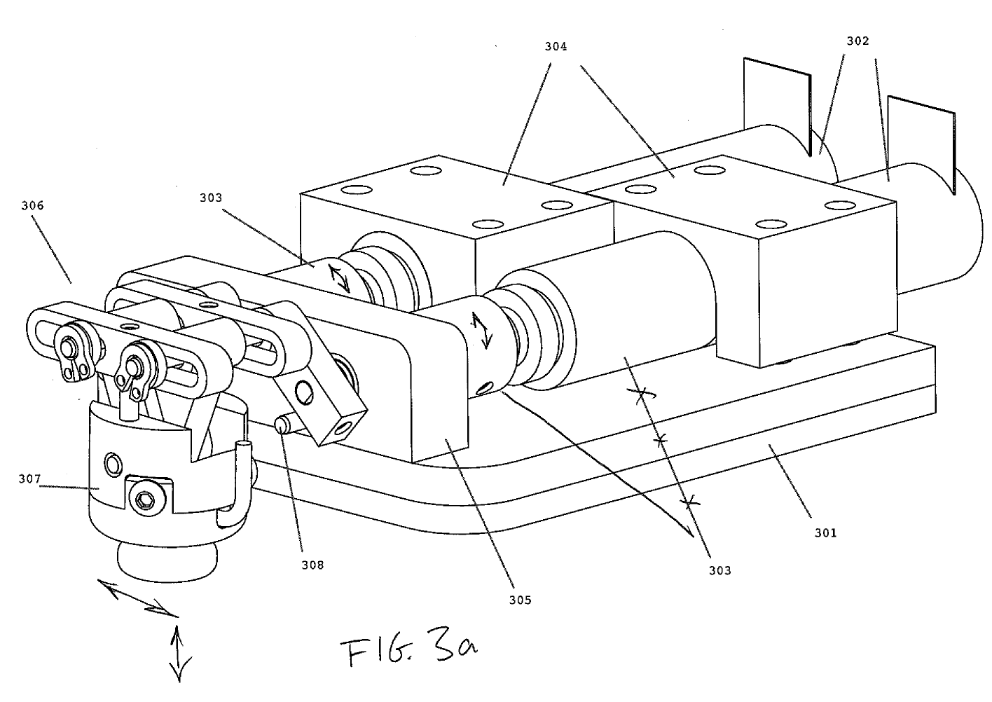

FIG. 2a details one exemplary embodiment of a detector. The base of the detector 201 includes a three-axis flexure 202 for a load cell, to which strain gauges (not shown) are adhered. In this embodiment three axes of force measurement are obtained, but other numbers are possible, more or fewer. In this embodiment the flexure is, advantageously, an inherent part of the detector base 201. However, a separately fabricated single or multi axis strain gauge can be substituted. Tip 203 is attached to the endpoint of flexure 202, by a pin 206 which engages a hole 207. Thus forces are transmitted reliably from the environment, via tip 203, to the flexure, and are measured by the strain gauges which are adhered to flexure 202. The strain gauges are not shown in the figure, nor are their wires, which exit via hole 208. Other known ways to measure forces, even multi-axis forces, such as optical measurements of deflection, magnetic measurements of deflection, or force sensitive conductive elastomers, can be used in place of the tips and flexure.

Tip 203 preferably includes other sensor and functional components. In a preferred embodiment the tip has the size and shape of a human fingertip and includes a hard stylus 205 which serves to elicit and transmit vibrations from a surface being explored by a wearer. Accelerometer 204 is attached to stylus 205 in order to best measure said vibrations as well as other accelerations due to contact and motion. Accelerometer 204 is, in the example, a two axis MEMS accelerometer in a preferred embodiment, but may have more or fewer active axes. A piezoelectric accelerometer may also be used, or other devices that are sensitive to vibration such as a magnetic pickup. Pins 209 and 210 serve as additional means of conveying vibration to the accelerometer.

FIGS. 2b and 2c illustrate an alternative embodiment of a receptor. Sensing element 211 is a two-axis flexure to which strain gages 212 are bonded. More or fewer axis of force measurement can be obtained by different embodiments of the flexure. The base of the sensing element attaches to a specially modified distal phalanx 213 of the prosthetic finger by means of screws 214. Accelerometer 215 rigidly attaches to sensing beam 211 by means of bracket 216. Sensing beam and accelerometer are encased in the fingertip cap 217 that attaches to the sensing beam 211 by means of screw 218 simultaneously retaining bracket 216 Modified distal phalanx 213 and cap 217 together comprise an anthropomorphic shape of a finger tip 222. A hard, fingernail-like stylus 219 attaches to cap 217 by means of screw 220 Pin 221 may be used as an alternative to the stylus. Electrical signals of the sensing beam and the accelerometer are transmitted to flexible circuit 219 by means of spring loaded pins in the base of the sensing element 211. Not shown is a tail of the flexible circuit that runs the length of the articulated finger to the palm of a prosthetic hand for housing sensor electronics. Although not illustrated, a temperature sensor or a thermal conductivity sensor, or a combination of the two measuring a combined quantity, can be included in an alternative embodiment. Other tactile, thermal, pressure, vibration, acceleration, or other measurement devices or arrays of such devices could be incorporated into the detector of the present invention, as well.

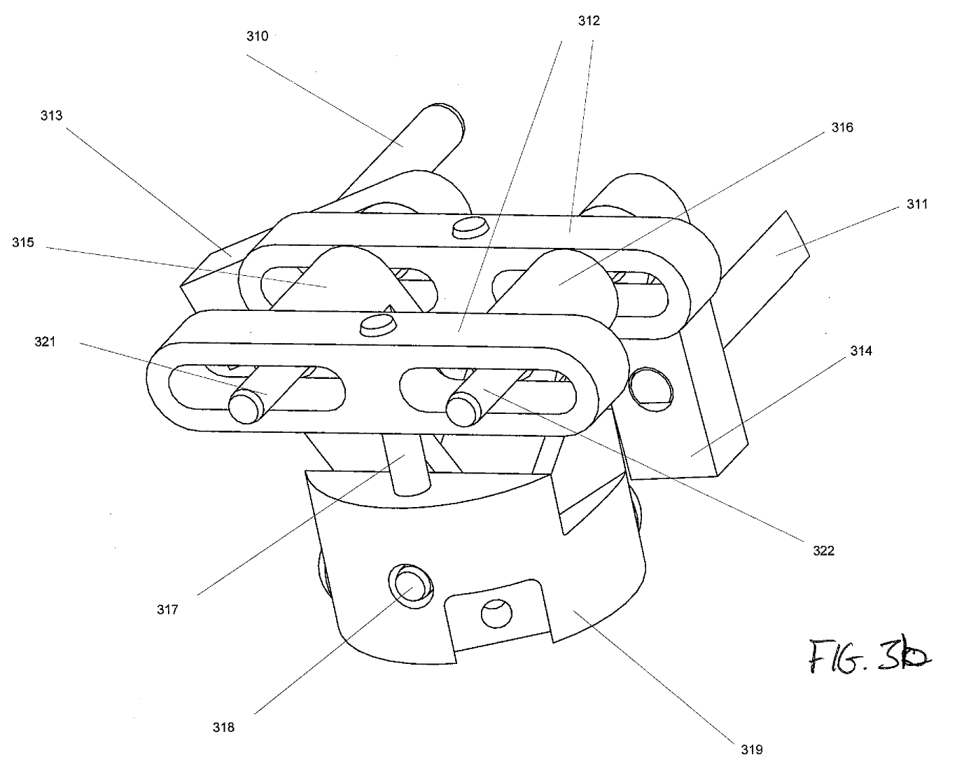

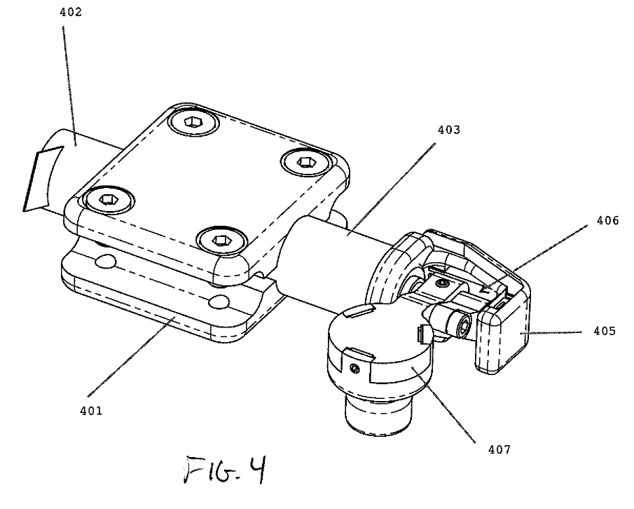

FIGS. 3a and 3b detail a preferred implementation of an exemplary stimulator. Base 301 attaches to pad 103 (see FIG. 1a) or to an adjustable component of socket 102. Motors 302 drive the axes of motion (two in this embodiment). Motors 302 are preferably DC brushless servomotors such as Maxon RE10. Other kinds of motors may also be used. Gear trains 303 provide higher torque than would motors 302 alone. Gear trains 303 are preferably low-backlash precision devices such as Maxon GP10A with a transmission ratio, in a preferred embodiment, of 16. Blocks 304 provide structural support and block 305, which holds rotational bearings, provides further structural support.

Linkage 306 serves to convert the rotational motion of the outputs of the two gear trains into approximately translational motions of the head of the stimulator 307. Linkage 306 can be described as a 5-bar mechanism with a prismatic constraint that prevents the stimulator tip, which is offset from the linkage pivot point, from tipping uncontrollably. In other words the additional prismatic linkage constraints the stimulator tip to a known orientation. Other numbers of axes of motion, more or less closely translational motion, could be used, and these would require other linkages. In this preferred embodiment there are two axes of approximately translational motion, and these are a motion perpendicular to the skin surface which may be used to transmit a sensation of pressure, and a motion parallel to the skin surface which may be used to transmit a sensation of shear. Pin 308 and another pin not visible limit the range of motion of the linkage in order to prevent excessive excursions.

Vibrations are transmitted by rapid modulation of the motions of the stimulator head 307. Stimulator head 307 preferably also incorporates a one axis force sensor measuring normal pressure of the stimulator head against the skin surface of the wearer. Alternatively, a two-axis or three-axis force sensor, which would measure shear forces as well, could be employed. As a further alternative, if no force sensor is used, contact with the skin could be measured. This could be done by a variety of techniques, including by measuring electrical conductivity. Stimulator head 307 could be integrated with a pad (not shown) for measuring EMG potentials. It could also incorporate a thermal heater and/or cooler (not shown), such as a Peltier device, to convey sensations of temperature to the wearer.

Referring now primarily to FIG. 3b, motor shafts 310 and 311 drive two axes of motion (two in this embodiment), and rigidly connect to cranks 313 and 314 respectively. Pins 321 and 322 are rigidly attached to the free ends of cranks 313 and 314 respectively. One end of link 315 freely rotates about pin 321, forming a pin connection to crank 313. Link 316 similarly rotates freely about pin 322, forming a pin connection to crank 314. The other end of each of links 315 and 316 attach to each other and to tactor load cell fixture 319 with a, shared, freely rotating, non-translating connection on pin 318, which is fixed in tactor load cell fixture 319. Tactor load cell fixture 319 is guided to remain centered between links 315 and 316 during mechanism motion, by an alignment mechanism consisting of two pins 317, and two tactor guide bars 312. Pins 317 are rigidly connected to, and axially aligned with, tactor load cell 319. Tactor guide bars 312 are free to rotate and translate on pins 317, allowing tactor guide bars 312 to move toward and away from tactor load cell fixture 319 during mechanism motion, while remaining perpendicular to load cell fixture 319. Slots in guide bars 312 are prismatically connected to pins 321 and 322, maintaining orientation of guide bars 312 and preserving centered angular relationship of tactor load cell fixture 319 with links 315 and 316.

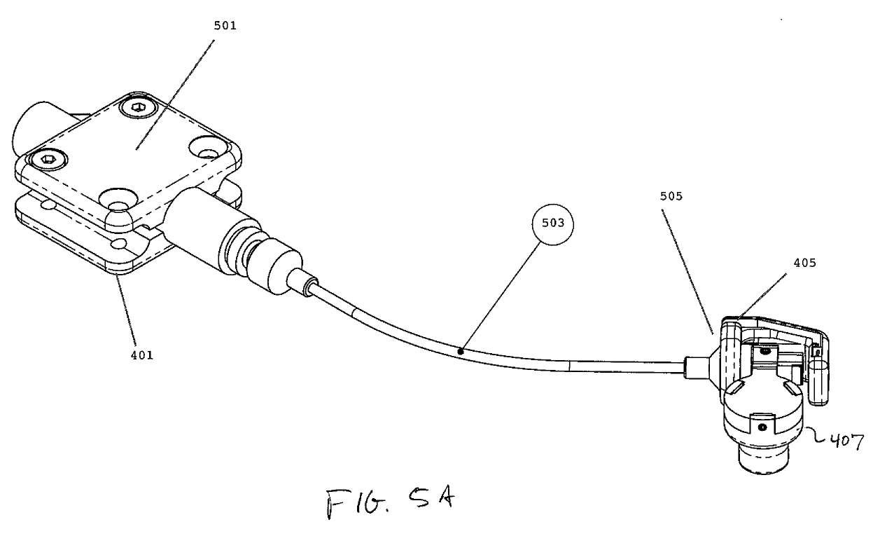

FIG. 4 details an alternate stimulator, differing primarily in that it produces only one axis of approximately translational motion, namely pressure. Base 401 is mountable to preloadable pad 103 (see FIG. 1b) or to an adjustable part of a socket. Motor 402 causes motion of the stimulator, and is preferably a DC brushless servomotor. Gear train 403 creates an increased torque, for driving linkage 406. Support structure 405 attaches to gear train 403 and carries linkage 406 which converts rotational motion of the output of gear train 403 into approximately translational motion of stimulator head 407. Stimulator head 407 is like stimulator head 307. All of the variations discussed for the stimulator embodiment of FIG. 3b, as well as additional ones not expressly discussed, may also be applied to this embodiment.

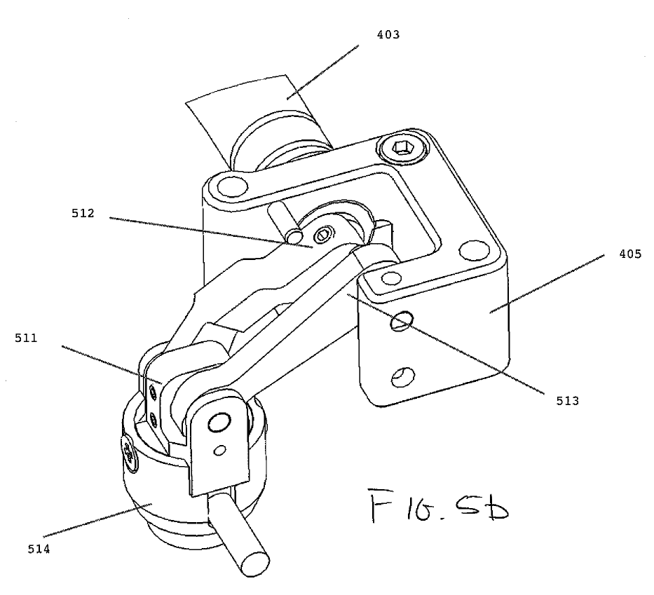

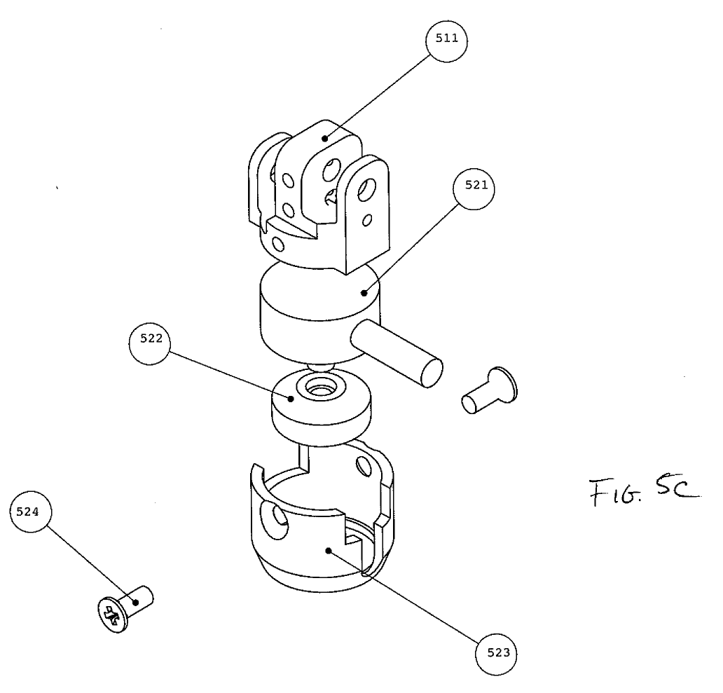

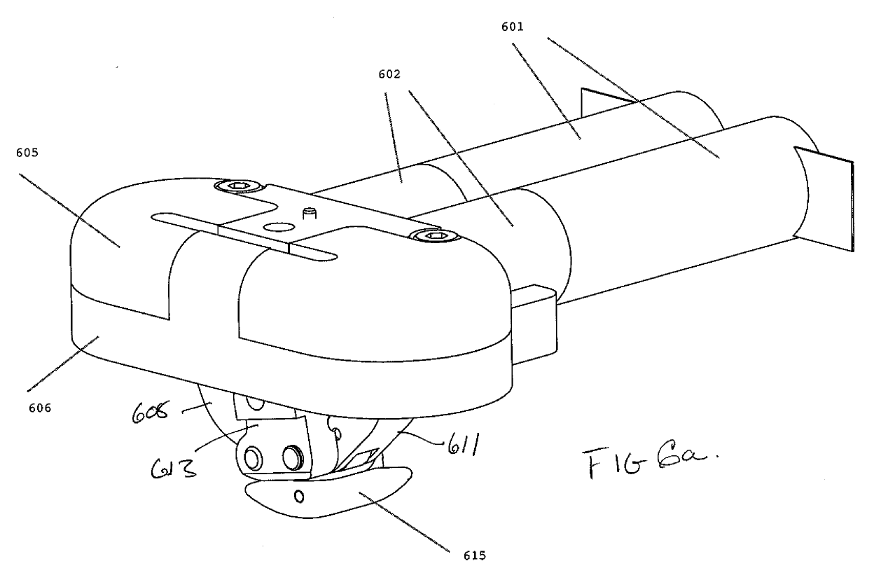

FIG. 5a illustrates, the interposition of a flexible shaft 503 that allows a stimulator to be divided into two parts, a stimulator actuator 501 and a stimulator endpart 505. In this example, the stimulator of FIG. 4 is illustrated. Other stimulators can also be used. Flexible shaft 503 allows rotation of an inner torsional component inside an outer housing. One example of a flexible shaft is the S. S. White Ready-Flex® flexible shaft. Alternately the torsional component may be sheathed. The separation of the two parts allows actuator 501 to be attached to a socket (e.g. socket 102 of FIG. 1b) and endpart 505 to be attached to an adjustable part of socket (e.g. pad 103 of FIG. 1b), which may move slightly with respect to the socket.

FIG. 5b further details a second embodiment of linkage 406. It is a 4-bar linkage. Arms 512 and 513 are rotatably attached to support structure 405 with parallel but non-coincident axes. Arms 512 and 513 are also rotatably attached to structure 511 with parallel but non-coincident axes. Thus structure 511 translates without rotation as arm 512 is turned by the output of gear train 403.

FIG. 5c further details stimulator head 514. It includes structure 511, previously described, a force sensor 521, a load distribution button 522, and a housing 523 attached by screw 524. The force sensor provided feedback to signal processors or other control circuitry for controlling the stimulator to absorb the amount of force actually being applied.

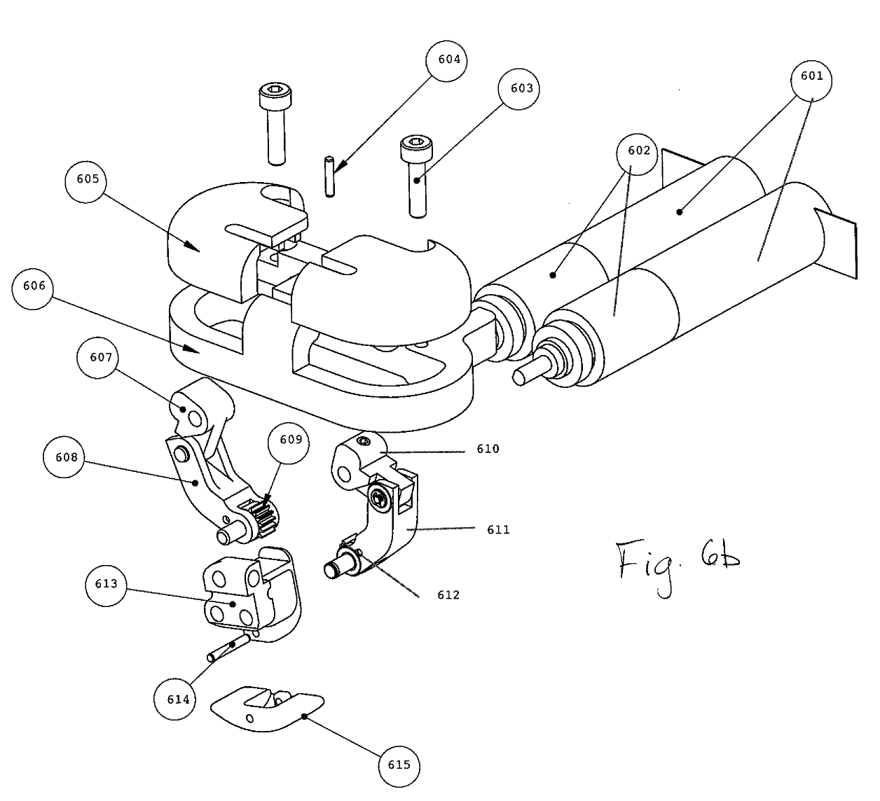

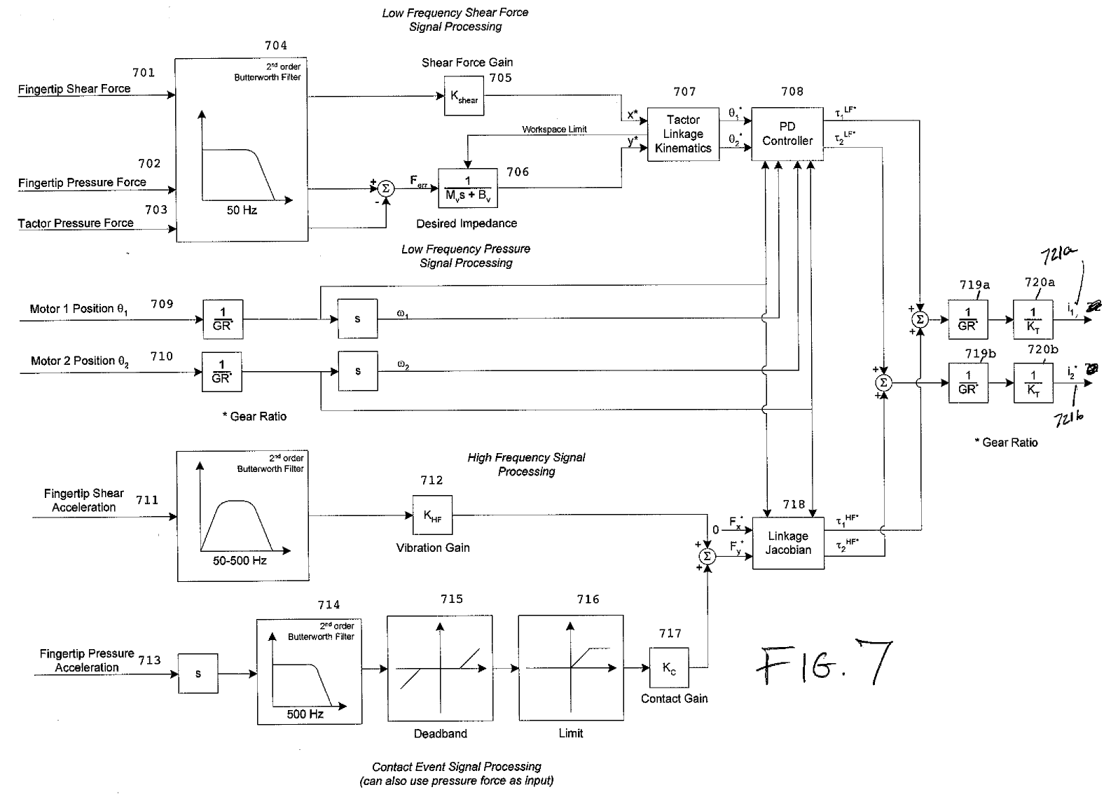

Referring to FIGS. 6a and 6b, an alternate embodiment of a two degree of freedom tactor. Motors 601 drive gear trains 602, which increase torque. A housing consisting of an upper part 605 and a lower part 606 contain a linkage which controls contact foot 615.

The linkage in the illustrated example is a 6-bar linkage. The outputs of gear trains 602 drive upper links 607 and 610, which are rotatably attached to lower links 608 and 611. Force sensor 613 is rotatably attached to lower links 608 and 611. Excess freedoms of force sensor 613 are removed by a gear pair 609 and 612 which engage with one another. Gears 609 and 612 are rigidly (non-rotatably) attached to lower links 608 and 611. Contact foot 615 is attached to end structure 613 by pin 614.

FIG. 7 schematically illustrates an exemplary implementation of signal processing that occurs between a detector and a stimulator such as those illustrated in the preceding figures. This is only an illustrative embodiment for a detector which provides two axes of force information and information from a two-axis accelerometer, one nominally normal and one tangential to the surface being touched, and for a stimulator which has two degrees of freedom. It also receives feedback from a single pressure force sensor on the stimulator. Each variation in the available signals from the detector and the degrees of freedom of the stimulator would require modification of the signal processing block diagram in FIG. 7. The processing occurs in signal processing circuitry, such as that described above.

Inputs 701 and 702 from the fingertip shear and pressure force sensors respectively, are filtered with a low pass filter 704, for example, a second order Butterworth filter with a 50 Hz cutoff. Feedback 703 from the stimulator pressure force sensor is also so filtered. The filtering can be done using analog or digital signals, or in software, using any known techniques. In this embodiment the fingertip shear force measurement 701, filtered as described, is multiplied by a gain 705 and combined with another axis of motion for pressure force display, originating in block 706. The output of block 706 is the difference between the fingertip pressure force measured 702 and the present stimulator pressure force measured 703, the difference constituting an error signal. The error signal is converted by impedance block 706 into a necessary corrective measurement. The two measurements are then converted by a linkage kinematics block 707 to motor rotation commands. The commands are conveyed to PD (proportional differential) controller block 708. PD controller 708 accepts also as inputs signals 709 and 710 indicating motor positions and their derivatives. The motor position signals are first multiplied by gear ratio blocks. The combinations are multiplied by gear ratio blocks 719 and 719b, and by motor constant blocks 720a and 720b, and thus become currents 721a and 721b with which to drive the motors.

Said other signals originate with the fingertip acceleration sensor, which provides a shear signal and a pressure signal. In a preferred embodiment these are processed differently, with fingertip shear acceleration signal 711 passing through a bandpass filter, for example a 2nd order Butterworth filter with bandpass frequencies 50 to 500 Hz, and then through a gain stage 712. Thus the high and low frequency components of motion of the stimulator originate separately in force sensors (for the low frequency components) and accelerometers (for the high frequency components). The resulting signal is then combined with fingertip pressure acceleration signal 713.

The fingertip pressure acceleration signal is, first, low-pass filtered by, for example, a 2nd order Butterworth filter with a cutoff frequency of 500 Hz, and then passed deadband filter 715, which has an adjustable threshold. It is then limited in magnitude by a limit filter 716 with an adjustable amplitude limit and by a contact gain factor 717. The signals are then combined, and converted into the motions needed by the axes of the two motors by linkage Jacobian calculations 718, which is informed by the linkage angles as derived from the motor angles 709 and 710. The output of the linkage Jacobian calculations, representing motor torques, are summed with the output of the PD controller 708. The combined motor torques, in the form of currents, are then passed to the motors as currents, as described previously.

What is claimed is:

1. (canceled)

2. An apparatus for providing afferent feedback from a prosthetic device, comprising:
a detector for mounting in a prosthetic device, the detector comprising a force sensor for sensing normal forces and shearing forces in at least one plane, the detector generating signals indicative of the normal and shearing forces;
a remotely located stimulator for engaging skin in order to provide afferent feedback based at least in part on the signals from the detector, the stimulator applying forces normal to the skin and along at least one axis tangential to the skin; the stimulator including an applicator mounted for moving along a first axis substantially normal to the skin and along at least a second axis tangential to the skin, the first and second axes being mutually orthogonal; the stimulator further including at least two, rotational actuators coupled with the applicator through a linkage for converting rotational motion for the actuators to movement of the applicator along each of the at least two-axes; and
signal processing circuitry for controlling the stimulator in response to the signals from the detector.

3. The apparatus of claim 2, wherein the linkage includes a 4-bar linkage.

4. The apparatus of claim 2, wherein the actuators are coupled through a drive gears for increasing torque.

5. The apparatus of claim 4 and wherein the linkage includes a 6-bar linkage.

6. The apparatus of claim 2, wherein the head includes a sensor for generating a feedback signal indicative of pressure on the contact element, the feedback signal communicating with the signal processing circuitry.

7. The apparatus of claim 2, wherein the stimulator is comprised of a head structure, an actuator support structure separate from the head structure, and a flexible shaft coupling rotational output of rotational actuators supported by the actuator support structure to the linkage supported by the head.

8. Apparatus for providing afferent feedback, comprising:
a detector comprising a force sensor for sensing normal forces and shearing forces along at least one axis; the detector comprising a multi-axis flexure element and at least one strain sensor for measuring deflection of the flexure element caused by application of the normal forces and the shearing forces, and generating signals indicative of the normal and shearing forces being at least in part responsive to the strain sensor;
a remotely located stimulator for engaging skin in order to provide afferent feedback based at least in part on the signals from the detector, the stimulator including a skin-engaging element for applying force normal to the skin and a force along at least one axis tangential to the skin; and
signal processing circuitry for controlling the stimulator in response to the signals from the detector.

9. The apparatus of claim 8, wherein the detector further comprises an anthropomorphically shaped force communicating structure coupled with the multi-axis flexure element.

10. The apparatus of claim 9, wherein the detector further comprises an accelerometer mounted to rigid, low mass element for detecting high frequency vibrations.

11. The apparatus of claim 8, wherein the stimulator is further comprised of a contact element mounted for moving along a first axis substantially normal to the skin and along at least a second axis tangential to the skin, the first and second axes being mutually orthogonal; the stimulator further including at least two, rotational actuators coupled with the head through a linkage for converting rotational motion for the actuators to movement of the head along each of the at least two axes, the rotational actuators being in communication with the signal processing circuitry.

12. The apparatus of claim 8, wherein the detector further includes a temperature sensor and the stimulator includes at least one thermal sensation unit for applying a heating and/or cooling sensation.

13. The apparatus of claim 12, wherein the thermal sensation unit is mounted in thermal communication with the skin-engaging element.

14. An apparatus for detecting and communicating tactile or haptic sensations, comprising:
an anthropomorphically-shaped force communicating structure;
a force sensor coupled with a force communicating structure, the detector mounted on a detector base for detecting static and low-frequency forces along at least 2 axes;
an accelerometer mounted on a rigid, low mass element for detecting higher frequency vibrations.

15. The detector of claim 14, wherein the force detector is comprised of a multi-axis flexure element coupled with the force detector, and at least one strain sensor for measuring deflection of the flexure element and generating in response the signals indicative of the forces applied along the axes.

16. The detector of claim 14, wherein the accelerometer is comprised of a MEMS device.

17. A stimulator for contacting skin for providing tactile sensation in response to actuation signals, comprising
a support structure;
an applicator mounted to the support structure for moving along a first axis substantially normal to the skin and along at least a second axis tangential to the skin, the first and second axes being mutually orthogonal;
at least two, rotational actuators coupled with the applicator through a linkage for converting rotational motion for the actuators to movement of the applicator along the at least two-axes.

18. The apparatus of claim 17, wherein the linkage includes a 4-bar linkage.

19. The apparatus of claim 17, wherein the actuators are coupled through a drive gears for increasing torque.

20. The apparatus of claim 17 and wherein the linkage includes a 6-bar linkage.

21. The apparatus of claim 17, wherein the head includes a sensor for generating a feedback signal indicative of pressure on the contact element, the feedback signal communicating with the signal processing circuitry.

22. The apparatus of claim 17, further comprising a flexible shaft coupling rotational output of rotational actuators to the applicator, the actuators being mounted on a structure separate from the support structure for the applicator.
